(19) United States
(12) Patent Application Publication
(10) Pub. No.: US 2008/0220530 A1
(43) Pub. Date: Sep. 11, 2008

(54) Biomarkers

(76) Inventors: Sabine Bahn, Cambridge (GB); Jeffrey T. Huang, Cambridge (GB); Tsz Tsang, London (GB)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/912,029

(22) PCT Filed: Jun. 5, 2006
(86) PCT No.: PCT/GB2006/050140

§ 371 Date: May 15, 2008

(30) Foreign Application Priority Data
Jun. 3, 2005 (GB) 0511302.2
Oct. 18, 2005 (GB) 0521098.4

(51) Int. Cl. G01N 33/00; C12Q 1/00

(52) U.S. Cl. 436/63; 435/4

(57) ABSTRACT

The invention relates to methods for diagnosing or monitoring psychotic disorders such as schizophrenic or bipolar disorders, comprising measuring the level of one or more biomarker(s) present in a cerebrospinal fluid sample taken from a test subject, said biomarker(s) being selected from the group consisting of: glucose, lactate, acetate species and pH. The invention also relates to methods of diagnosing or monitoring a psychotic disorder in a subject comprising providing a test sample of CSF from the subject, performing spectral analysis on said CSF test sample to provide one or more spectra, and, comparing the one or more spectra with one or more control spectra. The invention also relates to sensors, biosensors, multi-analyte panels, arrays, assays and kits for performing methods of the invention.

### TECHNICAL FIELD

The present invention relates to methods of diagnosing or of monitoring psychotic disorders, in particular schizophrenic disorders and bipolar disorders, using biomarkers. The biomarkers and methods in which they are employed can be used to assist diagnosis and to assess onset and development of psychotic disorders. The invention also relates to use of biomarkers in clinical screening, assessment of prognosis, evaluation of therapy, and for drug screening and drug development.

### BACKGROUND ART

The current diagnosis of psychotic conditions, such as schizophrenia and bipolar disorder, remains subjective, not only because of the complex spectrum of symptoms and their similarity to other mental disorders, but also due to the lack of empirical disease markers. There is a great clinical need for diagnostic tests and more effective drugs to treat severe mental illnesses.

Psychosis is a symptom of severe mental illness. Although it is not exclusively linked to any particular psychological or physical state, it is particularly associated with schizophrenia, bipolar disorder (manic depression) and severe clinical depression. Psychosis is characterized by disorders in basic perceptual, cognitive, affective and judgmental processes. Individuals experiencing a psychotic episode may experience hallucinations (often auditory or visual hallucinations), hold paranoid or delusional beliefs, experience personality changes and exhibit disorganised thinking (thought disorder). This is sometimes accompanied by features such as a lack of insight into the unusual or bizarre nature of their behaviour, difficulties with social interaction and impairments in carrying out the activities of daily living.

Psychosis is not uncommon in cases of brain injury and may occur after drug use, particularly after drug overdose or chronic use; certain compounds may be more likely to induce psychosis and some individuals may show greater sensitivity than others. The direct effects of hallucinogenic drugs are not usually classified as psychosis, as long as they abate when the drug is metabolised from the body. Chronic psychological stress is also known to precipitate psychotic states, however the exact mechanism is uncertain. Psychosis triggered by stress in the absence of any other mental illness is known as brief reactive psychosis. Psychosis is thus a descriptive term for a complex group of behaviours and experiences. Individuals with schizophrenia can have long periods without psychosis and those with bipolar disorder, or depression, can have mood symptoms without psychosis.

Hallucinations are defined as sensory perception in the absence of external stimuli. Psychotic hallucinations may occur in any of the five senses and can take on almost any form, which may include simple sensations (such as lights, colours, tastes, smells) to more meaningful experiences such as seeing and interacting with fully formed animals and people, hearing voices and complex tactile sensations. Auditory hallucination, particularly the experience of hearing voices, is a common and often prominent feature of psychosis. Hallucinated voices may talk about, or to the person, and may involve several speakers with distinct personas. Auditory hallucinations tend to be particularly distressing when they are derogatory, commanding or preoccupying.

Psychosis may involve delusional or paranoid beliefs, classified into primary and secondary types. Primary delusions are defined as arising out-of-the-blue and not being comprehensible in terms of normal mental processes, whereas secondary delusions may be understood as being influenced by the person's background or current situation, i.e. represent a delusional interpretation of a “real” situation.

Thought disorder describes an underlying disturbance to conscious thought and is classified largely by its effects on the content and form of speech and writing. Affected persons may also show pressure of speech (speaking incessantly and quickly), derailment or flight of ideas (switching topic mid-sentence or inappropriately), thought blocking, rhyming or punning.

Psychotic episodes may vary in duration between individuals. In brief reactive psychosis, the psychotic episode is commonly related directly to a specific stressful life event, so patients spontaneously recover normal functioning, usually within two weeks. In some rare cases, individuals may remain in a state of full blown psychosis for many years, or perhaps have attenuated psychotic symptoms (such as low intensity hallucinations) present at most times.

Patients who suffer a brief psychotic episode may have many of the same symptoms as a person who is psychotic as a result of (for example) schizophrenia, and this fact has been used to support the notion that psychosis is primarily a breakdown in some specific biological system in the brain.

Schizophrenia is a major psychotic disorder affecting up to 1% of the population. It is found at similar prevalence in both sexes and is found throughout diverse cultures and geographic areas. The World Health Organization found schizophrenia to be the world's fourth leading cause of disability that accounts for 1.1% of the total DALYs (Disability Adjusted Life Years) and 2.8% of YLDs (years of life lived with disability). It was estimated that the economic cost of schizophrenia exceeded US$ 19 billion in 1991, more than the total cost of all cancers in the United States. Effective treatments used early in the course of schizophrenia can improve prognosis and help reduce the costs associated with this illness.

The clinical syndrome of schizophrenia comprises discrete clinical features including positive symptoms (hallucination, delusions, disorganization of thought and bizarre behaviour); negative symptoms (loss of motivation, restricted range of emotional experience and expression and reduced hedonic capacity); and cognitive impairments with extensive variation between individuals. No single symptom is unique to schizophrenia and/or is present in every case. Despite the lack of homogeneity of clinical symptoms, the current diagnosis and classification of schizophrenia is still based on the clinical symptoms presented by a patient. This is primarily because the aetiology of schizophrenia remains unknown (in fact, the aetiology of most psychiatric diseases is still unclear) and classification based on aetiology is as yet not feasible. The clinical symptoms of schizophrenia are often similar to symptoms observed in other neuropsychiatric and neurodevelopmental disorders.

Due to the complex spectrum of symptoms presented by subjects with schizophrenic disorders and their similarity to other mental disorders, current diagnosis of schizophrenia is made on the basis of a complicated clinical examination/interview of the patient's family history, personal history, current symptoms (mental state examination) and the presence/absence of other disorders. This assessment allows a “most likely” diagnosis to be established, leading to the initial treatment plan. To be diagnosed with schizophrenia, a patient (with few exceptions) should have psychotic, “loss-of-reality” symptoms for at least six months (DSM IV) and show increasing difficulty in functioning normally.

The ICD-10 Classification of Mental and Behavioural Disorders, published by the World Health Organization in 1992, is the manual most commonly used by European psychiatrists to diagnose mental health conditions. The manual provides detailed diagnostic guidelines and defines the various forms of schizophrenia: schizophrenia, paranoid schizophrenia, hebrephrenic schizophrenia, catatonic schizophrenia, undifferentiated schizophrenia, post-schizophrenic schizophrenia, residual schizophrenia and simple schizophrenia.

The Diagnostic and Statistical Manual of Mental Disorders fourth edition (DSM IV) published by the American Psychiatric Association, Washington D.C., 1994, has proven to be an authoritative reference handbook for health professionals both in the United Kingdom and in the United States for categorising and diagnosing mental health problems. This describes the diagnostic criteria, subtypes, associated features and criteria for differential diagnosis of mental health disorders, including schizophrenia, bipolar disorder and related psychotic disorders.

### DSM IV Diagnostic Criteria for Schizophrenia

A. Characteristic symptoms: Two (or more) of the following, each present for a significant portion of time during a 1-month period (or less if successfully treated): delusions, hallucinations, disorganized speech (e.g., frequent derailment or incoherence), grossly disorganized or catatonic behaviour, negative symptoms, i.e., affective flattening, alogia, or avolition. Only one Criterion A symptom is required if delusions are bizarre or hallucinations consist of a voice keeping up a running commentary on the person's behaviour or thoughts, or two or more voices conversing with each other.

B. Social/occupational dysfunction: For a significant portion of the time since the onset of the disturbance, one or more major areas of functioning such as work, interpersonal relations, or self-care are markedly below the level achieved prior to the onset (or when the onset is in childhood or adolescence, failure to achieve expected level of interpersonal, academic, or occupational achievement).

C. Duration: Continuous signs of the disturbance persist for at least 6 months. This 6-month period must include at least 1 month of symptoms (or less if successfully treated) that meet Criterion A (i.e., active-phase symptoms) and may include periods of prodromal or residual symptoms. During these prodromal or residual periods, the signs of the disturbance may be manifested by only negative symptoms or two or more symptoms listed in Criterion A present in an attenuated form (e.g., odd beliefs, unusual perceptual experiences).

D. Schizoaffective and Mood Disorder exclusion: Schizoaffective Disorder and Mood Disorder With Psychotic Features have been ruled out because either (1) no Major Depressive Episode, Manic Episode, or Mixed Episode have occurred concurrently with the active-phase symptoms; or (2) if mood episodes have occurred during active-phase symptoms, their total duration has been brief relative to the duration of the active and residual periods.

E. Substance/general medical condition exclusion: The disturbance is not due to the direct physiological effects of a substance (e.g., a drug of abuse, a medication) or a general medical condition, so-called “organic” brain disorders/syndromes.

F. Relationship to a Pervasive Developmental Disorder: If there is a history of Autistic Disorder or another Pervasive Developmental Disorder, the additional diagnosis of Schizophrenia is made only if prominent delusions or hallucinations are also present for at least a month (or less if successfully treated).

### Schizophrenia Subtypes

1. Paranoid Type: A type of Schizophrenia in which the following criteria are met: preoccupation with one or more delusions (especially with persecutory content) or frequent auditory hallucinations. None of the following is prominent: disorganized speech, disorganized or catatonic behaviour, or flat or inappropriate affect.

2. Catatonic Type: A type of Schizophrenia in which the clinical picture is dominated by at least two of the following: motoric immobility as evidenced by catalepsy (including waxy flexibility) or stupor excessive motor activity (that is apparently purposeless and not influenced by external stimuli), extreme negativism (an apparently motiveless resistance to all instructions or maintenance of a rigid posture against attempts to be moved) or mutism, peculiarities of voluntary movement as evidenced by posturing (voluntary assumption of inappropriate or bizarre postures), stereotyped movements, prominent mannerisms, or prominent grimacing echolalia or echopraxia.

3. Disorganized Type: A type of Schizophrenia in which the following criteria are met: all of the following are prominent: disorganized speech, disorganized behaviour, flat or inappropriate affect. The criteria are not met for the Catatonic Type.

4. Undifferentiated Type: A type of Schizophrenia in which symptoms that meet Criterion A are present, but the criteria are not met for the Paranoid, Disorganized, or Catatonic Type.

5. Residual Type: A type of Schizophrenia in which the following criteria are met: absence of prominent delusions, hallucinations, disorganized speech, and grossly disorganized or catatonic behaviour. There is continuing evidence of the disturbance, as indicated by the presence of negative symptoms or two or more symptoms listed in Criterion A for Schizophrenia, present in an attenuated form (e.g., odd beliefs, unusual perceptual experiences).

### Schizophrenia Associated Features

Features associated with schizophrenia include: learning problems, hypoactivity, psychosis, euphoric mood, depressed mood, somatic or sexual dysfunction, hyperactivity, guilt or obsession, sexually deviant behaviour, odd/eccentric or suspicious personality, anxious or fearful or dependent personality, dramatic or erratic or antisocial personality.

Many disorders have similar or even the same symptoms as schizophrenia: psychotic disorder due to a general medical condition, delirium, or dementia; substance-induced psychotic disorder; substance-induced delirium; substance-induced persisting dementia; substance-related disorders; mood disorder with psychotic features; schizoaffective disorder; depressive disorder not otherwise specified; bipolar disorder not otherwise specified; mood disorder with catatonic features; schizophreniform disorder; brief psychotic disorder; delusional disorder; psychotic disorder not otherwise specified; pervasive developmental disorders (e.g., autistic disorder); childhood presentations combining disorganized speech (from a communication disorder) and disorganized behaviour (from attention-deficit/hyperactivity disorder); schizotypal disorder; schizoid personality disorder and paranoid personality disorder.

### DSM IV Diagnostic Categories for Manic Depression/Bipolar Affective Disorder (BD)

Only two sub-types of bipolar illness have been defined clearly enough to be given their own DSM categories, Bipolar I and Bipolar II.

Bipolar I: This disorder is characterized by manic episodes; the ‘high’ of the manic-depressive cycle. Generally this manic period is followed by a period of depression, although some bipolar I individuals may not experience a major depressive episode. Mixed states, where both manic or hypomanic symptoms and depressive symptoms occur at the same time, also occur frequently with bipolar I patients (for example, depression with the racing thoughts of mania). Also, dysphoric mania is common, this is mania characterized by anger and irritability.

Bipolar II: This disorder is characterized by major depressive episodes alternating with episodes of hypomania, a milder form of mania, Hypomanic episodes can be a less disruptive form of mania and may be characterized by low-level, non-psychotic symptoms of mania, such as increased energy or a more elated mood than usual. It may not affect an individual's ability to function on a day to day basis. The criteria for hypomania differ from those for mania only by their shorter duration (at least 4 days instead of 1 week) and milder severity (no marked impairment of functioning, hospitalization or psychotic features).

If alternating episodes of depressive and manic symptoms last for two years and do not meet the criteria for a major depressive or a manic episode then the diagnosis is classified as a Cyclothymic disorder, which is a less severe form of bipolar affective disorder. Cyclothymic disorder is diagnosed over the course of two years and is characterized by frequent short periods of hypomania and depressive symptoms separated by periods of stability.

Rapid cycling occurs when an individual's mood fluctuates from depression to hypomania or mania in rapid succession with little or no periods of stability in between. One is said to experience rapid cycling when one has had four or more episodes, in a given year, that meet criteria for major depressive, manic, mixed or hypomanic episodes. Some people who rapid cycle can experience monthly, weekly or even daily shifts in polarity (sometimes called ultra rapid cycling).

When symptoms of mania, depression, mixed mood, or hypomania are caused directly by a medical disorder, such as thyroid disease or a stroke, the current diagnosis is Mood Disorder Due to a General Medical Condition.

If a manic mood is brought about through an antidepressant, ECT or through an individual using “street” drugs, the diagnosis is Substance-induced Mood Disorder, with Manic Features.

Diagnosis of Bipolar III has been used to categorise manic episodes which occur as a result of taking an antidepressant medication, rather than occurring spontaneously. Confusingly, it has also been used in instances where an individual experiences hypomania or cyclothymia (i.e. less severe mania) without major depression.

### Mania

Manic Depression is comprised of two distinct and opposite states of mood, whereby depression alternates with mania. The DSM IV gives a number of criteria that must be met before a disorder is classified as mania. The first one is that an individual's mood must be elevated, expansive or irritable. The mood must be a different one to the individual's usual affective state during a period of stability. There must be a marked change over a significant period of time. The person must become very elevated and have grandiose ideas. They may also become very irritated and may well appear to be ‘arrogant’ in manner. The second main criterion for mania emphasizes that at least three of the following symptoms must have been present to a significant degree: inflated sense of self importance, decreased need for sleep, increased talkativeness, flight of ideas or racing thoughts, easily distracted, increased goal-directed activity. Excessive involvement in activities that can bring pleasure but may have disastrous consequences (e.g. sexual affairs and spending excessively). The third criterion for mania in the DSM IV emphasizes that the change in mood must be marked enough to affect an individual's job performance or ability to take part in regular social activities or relationships with others. This third criterion is used to emphasize the difference between mania and hypomania.

### Depression

The DSM IV states that there are a number of criteria by which major depression is clinically defined. The condition must have been evident for at least two weeks and must have five of the following symptoms: a depressed mood for most of the day, almost every day, a loss of interest or pleasure in almost all activities, almost every day, changes in weight and appetite, sleep disturbance, a decrease in physical activity, fatigue and loss of energy, feelings of worthlessness or excessive feelings of guilt, poor concentration levels, suicidal thoughts.

Both the depressed mood and a loss of interest in everyday activities must be evident as two of the five symptoms which characterize a major depression. It is difficult to distinguish between the symptoms of an individual suffering from the depressed mood of manic depression and someone suffering from a major depression. Dysthymia is a less severe depression than unipolar depression, but it can be more persistent.

The prolonged process currently needed to achieve accurate diagnosis of psychotic disorders may cause delay of appropriate treatment, which is likely to have serious implications for medium to long-term disease outcome. The development of objective diagnostic methods, tests and tools is urgently required to help distinguish between psychiatric diseases with similar clinical symptoms. Objective diagnostic methods and tests for psychotic disorders, such as schizophrenia and/or bipolar disorder, will assist in monitoring individuals over the course of illness (treatment response, compliance etc.) and may also be useful in determining prognosis, as well as providing tools for drug screening and drug development.

Unfortunately, at present there are no standard, sensitive, specific tests for psychotic disorders, such as schizophrenia or bipolar disorders.

One biochemical test currently under development for schizophrenia diagnosis is the niacin skin flush test, based on the observation that there is failure to respond to the niacin skin test in some schizophrenia patients, due to abnormal arachidonic acid metabolism. However, the specificity and sensitivity of this test shows an extreme inconsistency between studies, ranging from 23% to 87%, suggesting that the reliability and validity of this test still need to be verified.

International Patent Application Publication No. WO 01/63295 describes methods and compositions for screening, diagnosis, and determining prognosis of neuropsychiatric or neurological conditions (including BAD (bipolar affective disorder), schizophrenia and vascular dementia), for monitoring the effectiveness of treatment in these conditions and for use in drug development.

Other techniques such as magnetic resonance imaging or positron emission tomography based on subtle changes of the frontal and temporal lobes and the basal ganglia are of little value for the diagnosis, treatment, or prognosis of schizophrenic disorders in individual patients, since the absolute size of these reported differences between individuals with schizophrenia and normal comparison subjects has been generally small, with notable overlap between the two groups. The role of these neuroimaging techniques is restricted largely to the exclusion of other conditions which may be accompanied by schizophrenic symptoms, such as brain tumours or haemorrhages.

Therefore, a need exists to identify sensitive and specific biomarkers for diagnosis and for monitoring psychotic disorders, such as schizophrenic or bipolar disorders in a living subject. Additionally, there is a clear need for methods, models, tests and tools for identification and assessment of existing and new therapeutic agents for the treatment of these disorders.

Biomarkers present in readily accessible body fluids, such as cerebrospinal fluid (CSF), serum, urine or saliva, will prove useful in diagnosis of psychotic disorders, aid in predicting and monitoring treatment response and compliance, and assist in identification of novel drug targets. Appropriate biomarkers are also important tools in development of new early or pre-symptomatic treatments designed to improve outcomes or to prevent pathology.

The validation of biomarkers that can detect early changes specifically correlated to reversal or progression of mental disorders is essential for monitoring and optimising interventions. Used as predictors, these biomarkers can help to identify high-risk individuals and disease sub-groups that may serve as target populations for chemo-intervention trials; whilst as surrogate endpoints, biomarkers have the potential for assessing the efficacy and cost effectiveness of preventative interventions at a speed which is not possible at present when the incidence of manifest mental disorder is used as the endpoint.

Metabonomic studies can be used to generate a characteristic pattern or “fingerprint” of the metabolic status of an individual. Metabonomic studies on biological samples, such as biofluids provide information on the biochemical status of the whole organism.

“Metabonomics” is conventionally defined as “the quantitative measurement of the multi-parametric metabolic response of living systems to pathophysiological stimuli or genetic modification”. Metabonomics has developed from the use of ¹H NMR spectroscopy to study the metabolic composition of biological samples: biofluids, cells, and tissues, and from studies utilising pattern recognition (PR), expert systems and other chemoinformatic tools to interpret and classify complex NMR-generated metabolic data sets and to extract useful biological information.

Biofluids often exhibit very minor changes in metabolite profile in response to external stimuli. Dietary, diurnal and hormonal variations may also influence biofluid compositions, and it is clearly important to differentiate these effects if correct biochemical inferences are to be drawn from their analysis. Biomarker information provided by NMR spectra of biofluids is very subtle, as hundreds of compounds representing many pathways can often be measured simultaneously.

¹H NMR spectra of biological samples provide a characteristic metabolic “fingerprint” or profile of the organism from which the sample was obtained for a range of biologically-important endogenous metabolites [1-5]. This metabolic profile is characteristically changed by a disease, disorder, toxic process, or xenobiotic (e.g. drug substance). Quantifiable differences in metabolite patterns in biological samples can give information and insight into the underlying molecular mechanisms of disease or disorder. In the evaluation of the effects of drugs, each compound or class of compound produces characteristic changes in the concentrations and patterns of endogenous metabolites in biological samples.

The metabolic changes can be characterised using automated computer programs which represent each metabolite measured in the biological sample as a co-ordinate in multi-dimensional space.

Metabonomic technology has been used to identify biomarkers of inborn errors of metabolism, liver and kidney disease, cardiovascular disease, insulin resistance and neurodegenerative disorders [3, 4, 6-9]. Although a wealth of disease studies have been performed on biofluids such as urine and plasma, relatively few metabolite profiling studies have been performed on CSF for the purposes of disease diagnosis and identification of key metabolites as biomarkers [10-15].

### DISCLOSURE OF THE INVENTION

In one aspect, the invention provides a method of diagnosing or monitoring a psychotic disorder in a subject comprising:

(a) providing a test biological sample from said subject,
(b) performing spectral analysis on said test biological sample to provide one or more spectra, and,
(c) comparing said one or more spectra with one or more control spectra.

Biological samples that may be tested in a method of the invention include whole blood, blood serum or plasma, urine, saliva, cerebrospinal fluid (CSF) or other bodily fluid (stool, tear fluid, synovial fluid, sputum), breath, e.g. as condensed breath, or an extract or purification therefrom, or dilution thereof. Biological samples also include tissue homogenates, tissue sections and biopsy specimens from a live subject, or taken post-mortem. The samples can be prepared, for example where appropriate diluted or concentrated, and stored in the usual manner.

In one embodiment, the invention provides a method of diagnosing or monitoring a psychotic disorder in a subject comprising:

(a) providing a test sample of CSF from said subject,
(b) performing spectral analysis on said CSF test sample to provide one or more spectra, and,
(c) comparing said one or more spectra with one or more control spectra.

Monitoring methods of the invention can be used to monitor onset, progression, stabilisation, amelioration and/or remission of a psychotic disorder.

The term “diagnosis” as used herein encompasses identification, confirmation, and/or characterisation of a psychotic disorder, in particular a schizophrenic disorder, bipolar disorder, related psychotic disorder, or predisposition thereto. By predisposition it is meant that a subject does not currently present with the disorder, but is liable to be affected by the disorder in time.

A psychotic disorder is a disorder in which psychosis is a recognised symptom, this includes neuropsychiatric (psychotic depression and other psychotic episodes) and neurodevelopmental disorders (especially Autistic spectrum disorders), neurodegenerative disorders, depression, mania, and in particular, schizophrenic disorders (paranoid, catatonic, disorganized, undifferentiated and residual schizophrenia) and bipolar disorders.

The term “biomarker” means a distinctive biological or biologically derived indicator of a process, event, or condition. Biomarkers can be used in methods of diagnosis (e.g. clinical screening), prognosis assessment; in monitoring the results of therapy, identifying patients most likely to respond to a particular therapeutic treatment, in drug screening and development. Biomarkers are valuable for use in identification of new drug treatments and for discovery of new targets for drug treatment.

A number of spectroscopic techniques can be used to generate the spectra, including NMR spectroscopy and mass spectrometry. In preferred methods, spectral analysis is performed by NMR spectroscopy, preferably ¹H NMR spectroscopy. One or more spectra may be generated, a suite of spectra (i.e., multiple spectra) may be measured, including one for small molecules and another for macromolecule profiles. The spectra obtained may be subjected to spectral editing techniques. One or two-dimensional NMR spectroscopy may be performed.

An advantage of using NMR spectroscopy to study complex biomixtures is that measurements can often be made with minimal sample preparation (usually with only the addition of 5-10% D₂O) and a detailed analytical profile can be obtained on the whole biological sample.

Sample volumes are small, typically 0.3 to 0.5 ml for standard probes, and as low as 3 μl for microprobes. Acquisition of simple NMR spectra is rapid and efficient using flow-injection technology. It is usually necessary to suppress the water NMR resonance.

High resolution NMR spectroscopy (in particular ¹H NMR) is particularly appropriate. The main advantages of using ¹H NMR spectroscopy are the speed of the method (with spectra being obtained in 5 to 10 minutes), the requirement for minimal sample preparation, and the fact that it provides a non-selective detector for all metabolites in the biofluid regardless of their structural type, provided only that they are present above the detection limit of the NMR experiment and that they contain non-exchangeable hydrogen atoms.

NMR studies of biological samples, e.g. body fluids, should ideally be performed at the highest magnetic field available to obtain maximal dispersion and sensitivity and most ¹H NMR studies are performed at 400 MHz or greater, e.g. 600 MHz.

Usually, to assign ¹H NMR spectra, comparison is made with control spectra of authentic materials and/or by standard addition of an authentic reference standard to the sample. The control spectra employed may be normal control spectra, generated by spectral analysis of a biological sample (e.g., a CSF sample) from a normal subject, and/or psychotic disorder control spectra, generated by spectral analysis of a biological sample, (e.g., a CSF sample), from a subject with a psychotic disorder.

Additional confirmation of assignments is usually sought from the application of other NMR methods, including, for example, 2-dimensional (2D) NMR methods, particularly COSY (correlation spectroscopy), TOCSY (total correlation spectroscopy), inverse-detected heteronuclear correlation methods such as HMBC (heteronuclear multiple bond correlation), HSQC (heteronuclear single quantum coherence), and HMQC (heteronuclear multiple quantum coherence), 2D J-resolved (JRES) methods, spin-echo methods, relaxation editing, diffusion editing (e.g., both 1D NMR and 2D NMR such as diffusion-edited TOCSY), and multiple quantum filtering.

By comparison of spectra with normal and/or psychotic disorder control spectra, the test spectra can be classified as having a normal profile and or a psychotic disorder profile.

Comparison of spectra may be performed on entire spectra or on selected regions of spectra. Comparison of spectra may involve an assessment of the variation in spectral regions responsible for deviation from the normal spectral profile and in particular, assessment of variation in biomarkers within those regions.

A limiting factor in understanding the biochemical information from both 1D and 2D-NMR spectra of biofluids, such as CSF, is their complexity. Although the utility of the metabonomic approach is well established, its full potential has not yet been exploited. The metabolic variation is often subtle, and powerful analysis methods are required for detection of particular analytes, especially when the data (e.g., NMR spectra) are so complex. The most efficient way to compare and investigate these complex multiparametric data is employ the 1D and 2D NMR metabonomic approach in combination with computer-based “pattern recognition” (PR) methods and expert systems.

Metabonomics methods (which employ multivariate statistical analysis and pattern recognition (PR) techniques, and optionally data filtering techniques) of analysing data (e.g. NMR spectra) from a test population yield accurate mathematical models which may subsequently be used to classify a test sample or subject, and/or in diagnosis.

Comparison of spectra may include one or more chemometric analyses of the spectra. The term “chemometrics” is applied to describe the use of pattern recognition (PR) methods and related multivariate statistical approaches to chemical numerical data. Comparison may therefore comprise one or more pattern recognition analysis method(s), which can be performed by one or more supervised and/or unsupervised method(s).

Pattern recognition (PR) methods can be used to reduce the complexity of data sets, to generate scientific hypotheses and to test hypotheses. In general, the use of pattern recognition algorithms allows the identification, and, with some methods, the interpretation of some non-random behaviour in a complex system which can be obscured by noise or random variations in the parameters defining the system. Also, the number of parameters used can be very large such that visualisation of the regularities or irregularities, which for the human brain is best in no more than three dimensions, can be difficult.

Usually the number of measured descriptors is much greater than three and so simple scatter plots cannot be used to visualise any similarity or disparity between samples. Pattern recognition methods have been used widely to characterise many different types of problem ranging for example over linguistics, fingerprinting, chemistry and psychology.

In the context of the methods described herein, pattern recognition is the use of multivariate statistics, both parametric and non-parametric, to analyse spectroscopic data, and hence to classify samples and to predict the value of some dependent variable based on a range of observed measurements. There are two main approaches. One set of methods is termed “unsupervised” and these simply reduce data complexity in a rational way and also produce display plots which can be interpreted by the human eye. The other approach is termed “supervised” whereby a training set of samples with known class or outcome is used to produce a mathematical model and this is then evaluated with independent validation data sets.

Unsupervised techniques are used to establish whether any intrinsic clustering exists within a data set and consist of methods that map samples, often by dimension reduction, according to their properties, without reference to any other independent knowledge, e.g. without prior knowledge of sample class. Examples of unsupervised methods include principal component analysis (PCA), non-linear mapping (NLM) and clustering methods such as hierarchical cluster analysis.

One of the most useful and easily applied unsupervised PR techniques is principal components analysis (PCA) (see, for example, [40]). Principal components (PCs) are new variables created from linear combinations of the starting variables with appropriate weighting coefficients. The properties of these PCs are such that: (i) each PC is orthogonal to (uncorrelated with) all other PCs, and (ii) the first PC contains the largest part of the variance of the data set (information content) with subsequent PCs containing correspondingly smaller amounts of variance.

PCA, a dimension reduction technique, takes m objects or samples, each described by values in K dimensions (descriptor vectors), and extracts a set of eigenvectors, which are linear combinations of the descriptor vectors. The eigenvectors and eigenvalues are obtained by diagonalisation of the covariance matrix of the data. The eigenvectors can be thought of as a new set of orthogonal plotting axes, called principal components (PCs). The extraction of the systematic variations in the data is accomplished by projection and modelling of variance and covariance structure of the data matrix. The primary axis is a single eigenvector describing the largest variation in the data, and is termed principal component one (PC1). Subsequent PCs, ranked by decreasing eigenvalue, describe successively less variability. The variation in the data that has not been described by the PCs is called residual variance and signifies how well the model fits the data. The projections of the descriptor vectors onto the PCs are defined as scores, which reveal the relationships between the samples or objects. In a graphical representation (a “scores plot” or eigenvector projection), objects or samples having similar descriptor vectors will group together in clusters. Another graphical representation is called a loadings plot, and this connects the PCs to the individual descriptor vectors, and displays both the importance of each descriptor vector to the interpretation of a PC and the relationship among descriptor vectors in that PC. In fact, a loading value is simply the cosine of the angle which the original descriptor vector makes with the PC.

Descriptor vectors which fall close to the origin in this plot carry little information in the PC, while descriptor vectors distant from the origin (high loading) are important in interpretation.

Thus a plot of the first two or three PC scores gives the “best” representation, in terms of information content, of the data set in two or three dimensions, respectively. A plot of the first two principal component scores, PC1 and PC2 provides the maximum information content of the data in two dimensions. Such PC maps can be used to visualise inherent clustering behaviour, for example, for drugs and toxins based on similarity of their metabonomic responses and hence mechanism of action. Of course, the clustering information may be in lower PCs and these can also be examined.

Hierarchical Cluster Analysis, another unsupervised pattern recognition method, permits the grouping of data points which are similar by virtue of being “near” to one another in some multidimensional space. Individual data points may be, for example, the signal intensities for particular assigned peaks in an NMR spectrum. A “similarity matrix” S, is constructed with element ssij=1−rij/rijmax′ where rij is the interpoint distance between points i and j (e.g., Euclidean interpoint distance), and rijmax is the largest interpoint distance for all points.

The most distant pair of points will have sij equal to 0, since rij then equals rijmaX. Conversely, the closest pair of points will have the largest sij, approaching 1. The similarity matrix is scanned for the closest pair of points. The pair of points is reported with their separation distance, and then the two points are deleted and replaced with a single combined point. The process is then repeated iteratively until only one point remains. A number of different methods may be used to determine how two clusters will be joined, including the nearest neighbour method (also known as the single link method), the furthest neighbour method, the centroid method (including centroid link, incremental link, median link, group average link, and flexible link variations).

For two identical points, analysis of 300 samples per day per spectrometer is possible (with the first generation of flow injection systems), more subtle expert systems may be necessary, for example, using techniques such as “fuzzy logic” which permit greater flexibility in decision boundaries.

The reported connectivities can then be plotted as a dendrogram (a tree-like chart which allows visualisation of clustering), showing sample-sample connectivities versus increasing separation distance (or equivalently, versus decreasing similarity). In the dendrogram the branch lengths are proportional to the distances between the various clusters and hence the length of the branches linking one sample to the next is a measure of their similarity. In this way, similar data points may be identified algorithmically.

Supervised methods of analysis use the class information given for a training set of sample data to optimise the separation between two or more sample classes. These techniques include soft independent modelling of class analogy, partial least squares (PLS) methods, such as projection to latent discriminant analysis (PLS DA); k-nearest neighbour analysis and neural networks. Neural networks are a non-linear method of modelling data. A training set of data is used to develop algorithms that ‘learn’ the structure of the data and can cope with complex functions. Several types of neural network have been applied successfully to predicting toxicity or disease from spectral information.

Statistical techniques, such as one-way analysis of variance (ANOVA) or other statistical methods described herein, may also be employed to analyse data.

The invention further provides a method of diagnosing or monitoring a psychotic disorder in a subject comprising:

(a) providing a test biological sample from said subject,
(b) performing spectral analysis on said test biological sample to provide one or more spectra.
(c) analysing said one or more spectra to detect the level of one or more biomarkers in said spectra, and,
(d) comparing the level of said one or more biomarker(s) in said one or more spectra with the level of said one or more biomarker(s) detected in control spectra.

The invention yet further provides a method of diagnosing or monitoring a subject having a psychotic disorder comprising:

(a) providing a test sample of CSF from said subject,
(b) performing spectral analysis on said CSF test sample to provide one or more spectra,
(c) analysing said one or more spectra to detect the level of one or more biomarkers present in said one or more spectra, and,
(d) comparing the amount of said one or more biomarker(s) in said one or more spectra with one or more control spectra.

In particularly preferred methods, spectral analysis is performed by NMR spectroscopy, preferably ¹H NMR spectroscopy.

In methods of the invention involving spectral analysis, this may be performed to provide spectra from biological samples, such as CSF samples, taken on two or more occasions from a test subject. Spectra from biological samples taken on two or more occasions from a test subject can be compared to identify differences between the spectra of samples taken on different occasions. Methods may include analysis of spectra from biological samples, taken on two or more occasions from a test subject to quantify the level of one or more biomarker(s) present in the biological samples, and comparing the level of the one or more biomarker(s) present in samples taken on two or more occasions.

Diagnostic and monitoring methods of the invention are useful in methods of assessing prognosis of a psychotic disorder, in methods of monitoring efficacy of an administered therapeutic substance in a subject having, suspected of having, or of being predisposed to, a psychotic disorder and in methods of identifying an anti-psychotic or pro-psychotic substance. Such methods may comprise comparing the level of the one or more biomarker(s) in a biological sample, such as a CSF sample, taken from a test subject with the level present in one or more sample(s) taken from the test subject prior to administration of the substance, and/or one or more samples taken from the test subject at an earlier stage during treatment with the substance. Additionally, these methods may comprise detecting a change in the level of the one or more biomarker(s) in biological samples, such as CSF samples, taken from a test subject on two or more occasions.

In methods of the invention in which spectral analysis is employed, suitably one or more biomarker is selected from the group consisting of glucose, lactate, acetate (acetate species), alanine, glutamine or pH.

These biomarkers of psychotic disorder, in particular schizophrenic disorder, were identified by extensive metabolic profiling analysis of CSF samples from control and schizophrenia subjects using ¹H NMR spectroscopy in combination with computerised pattern recognition analysis. Significant differences in these biomarkers were found in samples obtained from first-onset, drug-naïve patients with a diagnosis of paranoid schizophrenia when compared to age-matched normal controls. In the group with psychotic disorder, the level of glucose in CSF was found to be higher than in CSF from normal individuals; serum glucose levels were not found to be elevated in individuals with psychotic disorder. The levels of lactate and acetate (acetylated species) were found to be lower in CSF from individuals with psychotic disorder when compared to the levels in CSF from normal subjects. The pH of CSF from subjects with psychotic disorder was found on average to be 0.1 units lower than the pH of CSF from normal individuals. This difference in pH resulted in a chemical shift in glutamine and alanine resonances. These differences constitute metabolic biomarkers in CSF that enable differentiation between normal individuals and those with a psychotic disorder.

In an further aspect, the invention provides a method of diagnosing or monitoring a psychotic disorder, or predisposition thereto, comprising measuring the level of one or more biomarker(s) present in a cerebrospinal fluid sample taken from a test subject, said biomarker being selected from the group consisting of: glucose, lactate, acetate species and pH. Such methods can be used in methods of monitoring efficacy of a therapy (e.g. a therapeutic substance) in a subject having, suspected of having, or of being predisposed to, a psychotic disorder.

Methods of diagnosing or monitoring according to the invention, may comprise measuring the level of one or more of the biomarker(s) present in CSF samples taken on two or more occasions from a test subject. Comparisons may be made between the level of biomarker(s) in samples taken on two or more occasions. Assessment of any change in the level of biomarker in samples taken on two or more occasions may be performed. Modulation of the biomarker level is useful as an indicator of the state of the psychotic disorder or predisposition thereto.

An increase in the level of glucose in CSF over time is indicative of onset or progression, i.e. worsening of the disorder, whereas a decrease in the level of glucose indicates amelioration or remission of the disorder.

A decrease in the level of lactate, acetylated species or pH in CSF over time is indicative of onset or progression, i.e. worsening of the disorder, whereas an increase in the level of these biomarkers indicates amelioration or remission of the disorder.

A method according to the invention may comprise comparing the level of one or more biomarker(s) in a CSF sample taken from a test subject with the level of the one or more biomarker(s) present in one or more sample(s) taken from the test subject prior to commencement of a therapy, and/or one or more sample(s) taken from the test subject at an earlier stage of a therapy. The level of a particular biomarker is compared with the level of the same biomarker in a different sample, i.e. congenic biomarkers are compared. Such methods may comprise detecting a change in the amount of the one or more biomarkers in samples taken on two or more occasions. Methods of the invention are particularly useful in assessment of anti-psychotic therapies, in particular in drug naïve subjects and in subjects experiencing their first psychotic episode. As described herein, using methods of the invention short-term treatment with atypical anti-psychotic medication was found to result in a normalization of the disease signature in half the patients who had been commenced on medication during their first psychotic episode, whilst those who had only been treated after several episodes did not show a normalization in CSF metabolite profile.

A method of diagnosis of or monitoring according to the invention may comprise quantifying the one or more biomarker(s) in a test CSF sample taken from a test subject and comparing the level of the one or more biomarker(s) present in said test sample with one or more controls. The control can be selected from a normal control and/or a psychotic disorder control. The control used in a method of the invention can be one or more control(s) selected from the group consisting of: the level of biomarker found in a normal control sample from a normal subject, a normal biomarker level; a normal biomarker range, the level in a sample from a subject with a schizophrenic disorder, bipolar disorder, related psychotic disorder, or a diagnosed predisposition thereto; a schizophrenic disorder marker level, a bipolar disorder marker level, a related psychotic disorder marker level, a schizophrenic disorder marker range, a bipolar disorder marker range and a related psychotic disorder marker range.

Biological samples such as CSF samples, can be taken at intervals over the remaining life, or a part thereof of a subject. Suitably, the time elapsed between taking samples from a subject undergoing diagnosis or monitoring will be 3 days, 5 days, a week, two weeks, a month, 2 months, 3 months, 6 or 12 months. Samples may be taken prior to and/or during and/or following an anti-psychotic therapy, such as an anti-schizophrenic or anti-bipolar disorder therapy.

Measurement of the level of a biomarker can be performed by any method suitable to identify the amount of the biomarker in a CSF sample taken from a patient or a purification of or extract from the sample or a dilution thereof. In methods of the invention, quantifying may be performed by measuring the concentration of the biomarker(s) in the sample or samples. In methods of the invention, in addition to measuring the concentration of the biomarker in CSF, the concentration of the biomarker may be tested in a different biological sample taken from the test subject, e.g. whole blood, blood serum, urine, saliva, or other bodily fluid (stool, tear fluid, synovial fluid, sputum), breath, e.g. as condensed breath, or an extract or purification therefrom, or dilution thereof. Biological samples also include tissue homogenates, tissue sections and biopsy specimens from a live subject, or taken post-mortem. The samples can be prepared, for example where appropriate diluted or concentrated, and stored in the usual manner.

Measuring the level of a biomarker present in a sample may include determining the concentration of the biomarker present in the sample, e.g. determining the concentration of one or more metabolite biomarker(s) selected from glucose, acetate (acetate species) and lactate. The concentration of hydrogen ions may be measured to provide the pH value of the sample. Such quantification may be performed directly on the sample, or indirectly on an extract therefrom, or on a dilution thereof.

For example, biomarker levels can be measured by one or more method(s) selected from the group consisting of: spectroscopy methods such as NMR (nuclear magnetic resonance), or mass spectroscopy (MS); SELDI (−TOF), MALDI (−TOF), a 1-D gel-based analysis, a 2-D gel-based analysis, liquid chromatography (e.g. high pressure liquid chromatography (HPLC) or low pressure liquid chromatography (LPLC)), thin-layer chromatography, and LC-MS-based techniques. Appropriate LC MS techniques include ICAT® (Applied Biosystems, CA, USA), or iTRAQ® (Applied Biosystems, CA, USA).

Measurement of a biomarker may be performed by a direct or indirect detected. method. A biomarker may be detected directly, or indirectly, via interaction with a ligand or ligands, such as an enzyme, binding receptor or transporter protein, peptide, aptamer, or oligonucleotide, or any synthetic chemical receptor or compound capable of specifically binding the biomarker. The ligand may possess a detectable label, such as a luminescent, fluorescent or radioactive label, and/or an affinity tag.

Metabolite biomarkers as described herein are suitably measured by conventional chemical or enzymatic methods (which may be direct or indirect and or may not be coupled), electrochemical, fluorimetric, luminometric, spectrophotometric, polarimetric, chromatographic (e.g. HPLC) or similar techniques.

For enzymatic methods consumption of a substrate in the reaction, or generation of a product of the reaction, may be detected, directly or indirectly, as a means of measurement.

Glucose can be detected and levels measured using various detection systems including conventional chemical agents, phenylboronic acids or other synthetic receptors, or enzymatic systems, such as single enzyme systems using, for example, glucose oxidase or glucose dehydrogenase (PQQ or NAD⁺); liquid chromatography, polarimetry, refractometry, spectrophotometric methods, fluorimetry, magnetic optical rotatory dispersion or near IR, and by specific binding to ligands such as lectins or transporter proteins.

Acetate species can be detected and levels measured using coupled enzymatic systems based on acetate kinase, pyruvate kinase and lactate dehydrogenase as described in Bergmeyer, I. U. (1983) Methods of Enzymatic Analysis, 3^{rd} ed., II, 127-128.

Lactate can be detected and levels measured using enzymatic systems, e.g. based on coupled enzyme systems incorporating lactate dehydrogenase or lactate oxidase/peroxidase.

The glucose, lactate and acetate biomarkers of the invention are preferably detected and measured using mass spectrometry-based techniques; chromatography-based techniques; enzymatic detection systems (by direct or indirect measurements); or using sensors, e.g. with sensor systems with amperometric, potentiometric, conductimetric, impedance, magnetic, optical, acoustic or thermal transducers.

A sensor may incorporate a physical, chemical or biological detection system, a biosensor is a sensor with a biological recognition system, e.g. based on an enzyme, receptor protein or nucleic acid.

Measurement of pH can be performed using glass or metal oxide electrodes, FETs or colorimetric/fluorimetric or luminescent measurement systems.

Methods of the invention are suitable for clinical screening, assessment of prognosis, monitoring the results of therapy, identifying patients most likely to respond to a particular therapeutic treatment, for drug screening and development, and to assist in identification of new targets for drug treatment. The identification of key biomarkers specific to a disease is central to integration of diagnostic procedures and therapeutic regimes. Using predictive biomarkers appropriate diagnostic tools such as sensors and biosensors can be developed, accordingly, in methods and uses of the invention, detecting and quantifying one or more biomarker(s) can be performed using a sensor or biosensor.

Biomarker levels may be detected using a sensor or biosensor, preferably a sensor or biosensor according to the invention is psychotic disorder sensor or biosensor capable of quantifying one, two, three or four biomarker(s) selected from the group: glucose, lactate, acetate and pH.

The sensor or biosensor may incorporate detection methods and systems as described herein for detection of the biomarker. Sensors or biosensors may employ electrical (e.g. amperometric, potentiometric, conductimetric, or impedance detection systems), thermal (e.g. transducers), magnetic, optical (e.g. hologram) or acoustic technologies. In a sensor or biosensor according to the invention the level of one, two, three or four biomarker(s) can be detected by one or more method selected from: direct, indirect or coupled enzymatic, spectrophotometric, fluorimetric, luminometric, spectrometric, polarimetric and chromatographic techniques. Particularly preferred sensors or biosensors comprise one or more enzyme(s) used directly or indirectly via a mediator, or using a binding, receptor or transporter protein, coupled to an electrical, optical, acoustic, magnetic or thermal transducer. Using such biosensors, it is possible to detect the level of target biomarker(s) at the anticipated concentrations found in biological samples.

A biomarker or biomarkers of the invention can be detected using a sensor or biosensor incorporating technologies based on “smart” holograms, or high frequency acoustic systems, such systems are particularly amenable to “bar code” or array configurations.

In smart hologram sensors (Smart Holograms Ltd, Cambridge, UK), a holographic image is stored in a thin polymer film that is sensitised to react specifically with the biomarker. On exposure, the biomarker reacts with the polymer leading to an alteration in the image displayed by the hologram. The test result read-out can be a change in the optical brightness, image, colour and/or position of the image. For qualitative and semi-quantitative applications, a sensor hologram can be read by eye, thus removing the need for detection equipment. A simple colour sensor can be used to read the signal when quantitative measurements are required. Opacity or colour of the sample does not interfere with operation of the sensor. The format of the sensor allows multiplexing for simultaneous detection of several substances. Reversible and irreversible sensors can be designed to meet different requirements, and continuous monitoring of a particular biomarker of interest is feasible.

Suitably, biosensors for detection of the biomarker of the invention are coupled, i.e. they combine biomolecular recognition with appropriate means to convert detection of the presence, or quantitation, of the biomarker in the sample into a signal. Biosensors can be adapted for “alternate site” diagnostic testing, e.g. in the ward, outpatients' department, surgery, home, field and workplace.

Biosensors to detect the biomarker(s) of the invention include acoustic, plasmon resonance, holographic and microengineered sensors. Imprinted recognition elements, thin film transistor technology, magnetic acoustic resonator devices and other novel acousto-electrical systems may be employed in biosensors for detection of the biomarker(s) of the invention.

Methods involving detection and/or quantification of a biomarker or biomarkers of the invention can be performed on bench-top instruments, or can be incorporated onto disposable, diagnostic or monitoring platforms that can be used in a non-laboratory environment, e.g. in the physician's office or at the patient's bedside. Suitable sensors or biosensors for performing methods of the invention include “credit” cards with optical or acoustic readers. Sensors or biosensors can be configured to allow the data collected to be electronically transmitted to the physician for interpretation and thus can form the basis for e-neuromedicine.

In methods of diagnosis and monitoring, a higher level of the glucose biomarker in the test CSF sample relative to the level in a normal control is indicative of the presence of a psychotic disorder, in particular a schizophrenic disorder, bipolar disorder, or predisposition thereto. An decrease in the level of glucose in the test CSF sample from an individual with a psychotic disorder, particular in individuals with a schizophrenic disorder, is indicative of absence or amelioration of the psychotic disorder.

In methods of diagnosis and monitoring, a lower level of one or more of the lactate, acetate species or pH biomarkers in the test CSF sample relative to the level in a normal control is indicative of the presence of a psychotic disorder, in particular a schizophrenic disorder, bipolar disorder, or predisposition thereto. A higher level of one or more of the lactate, acetate species or pH biomarkers in the test CSF sample relative to the level in a normal control is indicative of absence or amelioration of the psychotic disorder.

The pH associated shift in glutamine and alanine resonances away from the normal NMR spectral profile is indicative of the presence of a psychotic disorder, in particular a schizophrenic disorder, bipolar disorder, or predisposition thereto. A pH associated shift in glutamine and alanine resonances towards the normal NMR spectral profile is indicative of the absence or amelioration of a psychotic disorder, in particular a schizophrenic disorder, bipolar disorder, or predisposition thereto.

Methods of monitoring and of diagnosis according to the invention are useful to confirm the existence of a disorder, or predisposition thereto; to monitor development of the disorder by assessing onset and progression, or to assess amelioration or regression of the disorder. Methods of monitoring and of diagnosis are also useful in methods for assessment of clinical screening, prognosis, choice of therapy, evaluation of therapeutic benefit, i.e. for drug screening and drug development. These methods are particularly effective in drug naïve subjects and in those experiencing their first psychotic episode.

Efficient diagnosis and monitoring methods provide very powerful “patient solutions” with the potential for improved prognosis, by establishing the correct diagnosis, allowing rapid identification of the most appropriate treatment (thus lessening unnecessary exposure to harmful drug side effects), reducing “down-time” and relapse rates.

Methods for monitoring efficacy of a therapy can be used to monitor the therapeutic effectiveness of existing therapies and new therapies in human subjects and in non-human animals (e.g. in animal models). These monitoring methods can be incorporated into screens for new drug substances and combinations of substances.

In a further aspect the invention provides a multi-analyte panel or array capable of detecting one, two, three or four biomarker(s) selected from the group: glucose, acetate species, lactate, and pH.

A multi-analyte panel is capable of detecting a number of different analytes. An array can be capable of detecting a single analyte in a number of samples or, as a multi-analyte array, can be capable of detecting a number of different analytes in a sample. A multi-analyte panel or multi-analyte array according to the invention is capable of detecting one or more metabolic biomarker as described herein, and can be capable of detecting a biomarker or biomarkers additional to those specifically described herein.

Also provided is a diagnostic or monitoring test kit suitable for performing a method according to the invention, optionally together with instructions for use of the kit. The diagnostic or monitoring kit may comprise one or more biosensor(s) according to the invention, a single sensor, or biosensor or combination of sensor(s) and/or biosensors may be included in the kit. A diagnostic or monitoring kit may comprise a panel or an array according to the invention. A diagnostic or monitoring kit may comprise an assay or combination of assays for performing a method according to the invention.

Further provided is the use of one or more CSF biomarker(s) selected from glucose, lactate, acetate species, glutamine, alanine and pH to diagnose and/or monitor a psychotic disorder.

Yet further provided is the use of a method, sensor, biosensor, multi-analyte panel, array or kit according to the invention to identify a substance capable of modulating a psychotic disorder. A substance capable of modulating a psychotic disorder may be an anti psychotic substance useful for treatment of psychoses, or a pro-psychotic substance which may induce psychoses.

Additionally provided is a method of identifying a substance capable of modulating a psychotic disorder in a subject, comprising a method of monitoring as described herein; particularly preferred identification methods comprise administering a test substance to a test subject and detecting the level of one or more biomarker(s) selected from glucose, lactate, acetate species and pH in a CSF sample taken from said subject.

High-throughput screening technologies based on the biomarkers, uses and methods of the invention, e.g. configured in an array format, are suitable to monitor biomarkers for the identification of potentially useful therapeutic compounds, e.g. ligands such as natural compounds, synthetic chemical compounds (e.g. from combinatorial libraries), peptides, monoclonal or polyclonal antibodies or fragments thereof capable of modulating the biomarker.

Methods of the invention can be performed in multi-analyte panel or array format, e.g. on a chip, or as a multiwell array. Methods can be adapted into platforms for single tests, or multiple identical or multiple non-identical tests, and can be performed in high throughput format. Methods of the invention may comprise performing one or more additional, different tests to confirm or exclude diagnosis, and/or to further characterise a psychotic condition.

The identification of biomarkers for psychotic disorders, in particular schizophrenic disorders and bipolar disorders permits integration of diagnostic procedures and therapeutic regimes. Currently there are significant delays in determining effective treatment and it has not hitherto been possible to perform rapid assessments of drug response. Traditionally, many anti-psychotic therapies have required treatment trials lasting weeks to months for a given therapeutic approach. Detection of biomarkers of the invention can be used to screen subjects prior to their participation in clinical trials. The biomarkers provide the means to indicate therapeutic response, failure to respond, unfavourable side-effect profile, degree of medication compliance and achievement of adequate serum drug levels. The biomarkers may be used to provide warning of adverse drug response, a major problem encountered with all psychotropic medications. Biomarkers are useful in development of personalized brain therapies, as assessment of response can be used to fine-tune dosage, minimise the number of prescribed medications, reduce the delay in attaining effective therapy and avoid adverse drug reactions. Thus by monitoring biomarkers in accordance with the invention, patient care can be tailored precisely to match the needs determined by the disorder and the pharmacogenomic profile of the patient; the biomarker can thus be used to titrate the optimal dose, predict a positive therapeutic response and identify those patients at high risk of severe side effects.

Biomarker based tests provide a first line assessment of ‘new’ patients, and provide objective measures for accurate and rapid diagnosis, in a time frame and with precision, not achievable using the current subjective measures.

Furthermore, diagnostic biomarker tests are useful to identify family members or patients in the “prodromal phase”, i.e. those at high risk of developing overt schizophrenia, bipolar disorder, or related psychotic disorder. This permits initiation of appropriate therapy, for example low dose anti-psychotics, or preventive measures, e.g. managing risk factors such as stress, illicit drug use, or viral infections. These approaches are recognised to improve outcome and may prevent overt onset of the disorder.

Biomarker monitoring methods, sensors, biosensors and kits are also vital as patient monitoring tools, to enable the physician to determine whether relapse is due to a genuine breakthrough or worsening of the disease, poor patient compliance or substance abuse. If pharmacological treatment is assessed to be inadequate, then therapy can be reinstated or increased. For genuine breakthrough disease, a change in therapy can be given if appropriate. As the biomarker is sensitive to the state of the disorder, it provides an indication of the impact of drug therapy, or of substance abuse.

### LIST OF FIGURES

FIG. 1. Metabonomic analysis of CSF samples from drug-naïve schizophrenic patients.

(A) Partial ¹H NMR spectrum of a CSF sample from a representative drug-naïve schizophrenia patient (grey) and a matched control (black) illustrate a characteristic pH-dependent shift in the β-CH₂ and γ-CH₂ resonances of glutamine. The prominent signals at ˜3.7 and 1.2 ppm correspond to ethanol, a contaminant from skin disinfection prior to lumbar puncture. These signals were removed from statistical analysis.

(B) PLS-DA scores plot showing a differentiation of drug-naïve schizophrenia patients (triangles) from demographically matched healthy volunteer controls (squares) as determined by the ¹H NMR CSF spectra.

(C) PLS-DA loadings plot showing major contributing variables towards the separation in the PLS-DA scores plots.

FIG. 2. Effects of “typical” and “atypical” medication on CSF metabolic profiles in first onset schizophrenia patients.

(A) Spectra from a further 28 CSF samples from first onset schizophrenia patients minimally treated (<9 days) with either typical (n=6, diamonds) or atypical (n=22, circles) anti-psychotic medication and were compared to first onset, drug naïve schizophrenia patients (triangles) and healthy volunteers (squares) using PLS-DA models. The PLS-DA scores plots show that atypical anti-psychotic drug treatment resulted in a shift of approximately 50% of schizophrenia patients towards the cluster of healthy controls.

(B) The same PLS-DA scores plot as (A) except that only minimally treated patients (from both drug groups) with more than one psychotic episode prior to anti-psychotic treatment are shown. None of these patients shifted towards the healthy control cluster.

FIG. 3 Validation and prediction of schizophrenia group membership using a PLS model.

A PLS model was constructed using the OSC filtered data from 37 first onset, drug naïve schizophrenia patients (empty circles) and 50 healthy volunteers (filled circles) (the ‘training set’). The scores plot (A) and the loadings plot (B) indicate key resonances contributing to the separation: lactate, glucose, glutamine and citrate. This model was then used to predict “group membership” (i.e. disease or control) in a randomised test set of 17 first onset, drug naïve schizophrenia patients and 20 healthy volunteers which had not been used in the construction of the model. Predictions are made using a Y-predicted scatter plot with an a priori cut-off of 0.5 for class membership (C).

FIG. 4. Replication of metabonomic analysis on CSF samples from a “training sample set” comprising of 50 healthy volunteers and 37 first onset, drug naïve schizophrenia patients.

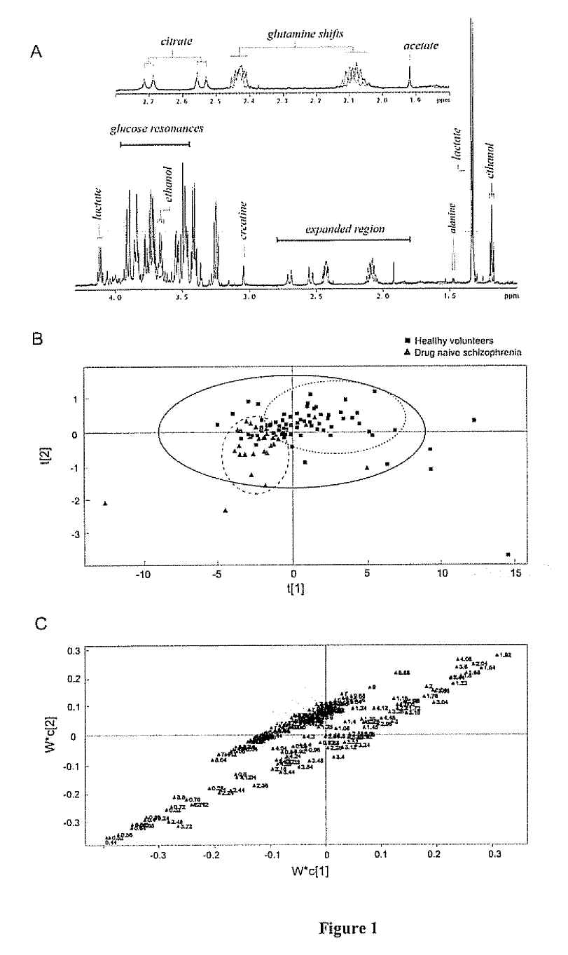

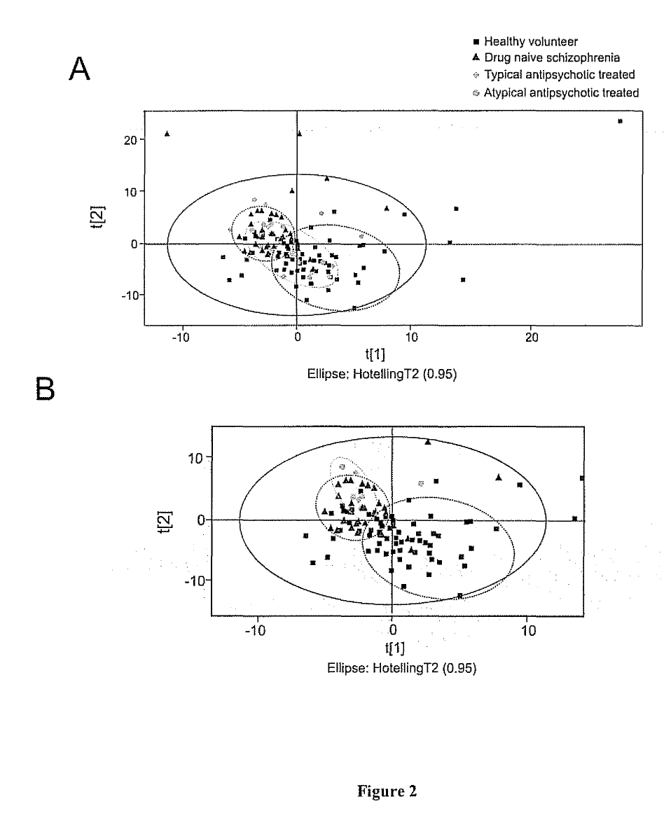

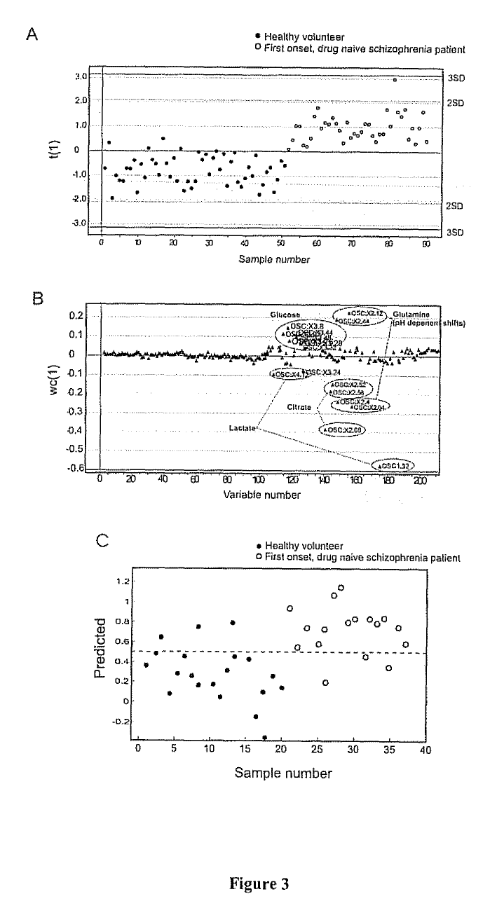

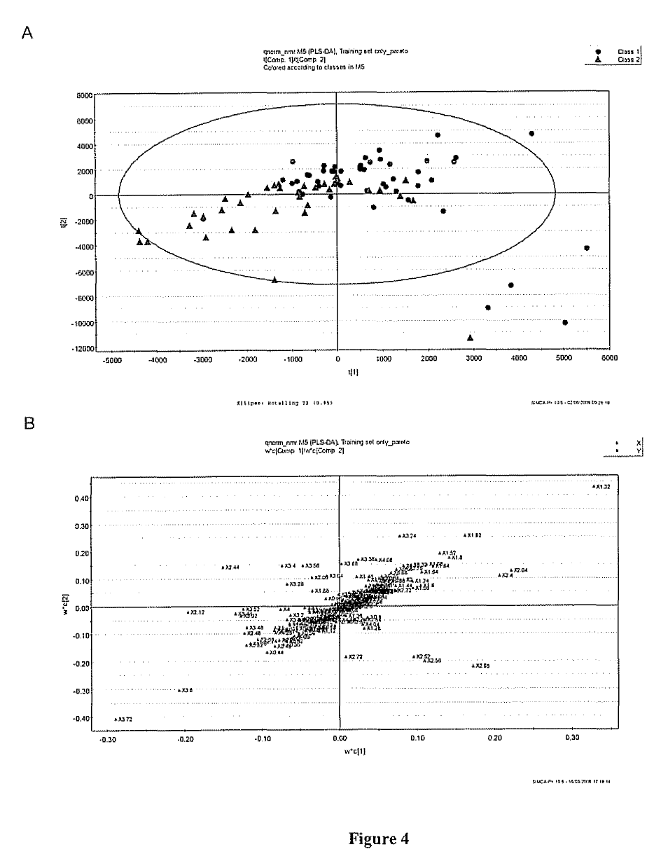

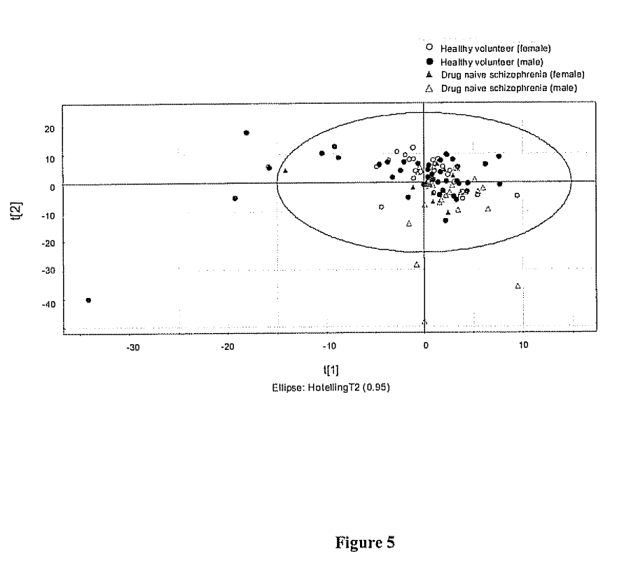

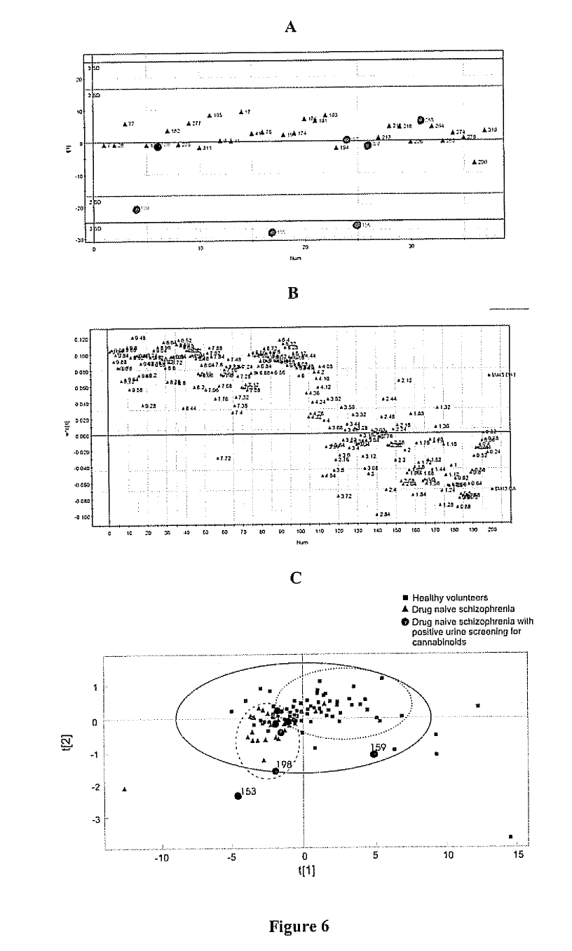

(A and B) PLS-DA scores and loadings plots show profiles and components discriminating between healthy volunteers () and drug naïve schizophrenia patients (▴), indicating a similar result as reported in FIG. 1. These samples were independently re-analyzed under an identical conditions. Note that the key variables are highly similar to those in FIG. 1.

FIG. 5. PLS-DA model demonstrating that gender did not influence the CSF metabolite profile in either healthy volunteers, nor in the drug naïve schizophrenia group. The symbols used are as follows: healthy volunteer female (empty circle), healthy volunteer male (filled circle); drug naïve schizophrenia female (filled triangle), drug naïve schizophrenia male (empty triangle).

FIG. 6. CSF metabolite profiles of schizophrenia patients who tested positive for cannabis on urine drug screen.

(A) and (B) PLS-DA scores plots showing profiles and discriminating components of cannabis positive vs. drug naïve, cannabis negative, schizophrenia patients (filled circles and triangles, respectively).

(C) Localisation of cannabis positive (circles) drug naïve schizophrenia patients in the PLS-DA plot in relation to healthy volunteers (squares) and drug naïve schizophrenia patients who tested negative for cannabis (triangles). Patients 153, 159 and 196 (all drug naïve schizophrenia patients with positive urine screening for cannabinoids) show a highly altered metabolite profile (A) and appear to form a separate cluster (C).

### EXAMPLES

The invention will be further understood by reference to the examples provided below.

### Methods and Materials

The Ethical committee of the Medical Faculty of the University of Cologne reviewed and approved the protocol of this study and the procedures for sample collection and analysis. All study participants gave their written informed consent. All clinical investigations were conducted according to the principles expressed in the Declaration of Helsinki. CSF samples were collected from drug-naïve patients diagnosed with first episode paranoid schizophrenia or brief psychotic disorder due to duration of illness (DSM-IV 295.30 or 298.8; n=54) and from demographically matched healthy volunteers (n=70) (Table 1). Additionally, samples from patients fulfilling DSM-IV criteria of schizophrenia (DSM-IV 295.30) undergoing treatment with either typical (total n=6: Haloperidol n=4, Perazine n=1, Fluphenazine n=1) or atypical (total n=22: Olanzapine n=9, Risperidone n=8, Quetiapine n=2, Amisulpride n=1, Clozapine n=1, Ziprasidone n=1) anti-psychotic medication were also included.

Due to an over-representation of females in the healthy volunteer group the effect of gender on the metabolite profile was examined, but no gender-specific effect was found (FIG. 5). The influence of recent and lifetime cannabis use was examined, determined by urine drug screen and clinical interview respectively (FIG. 6 and Table 2).

All samples were collected in a standardised fashion by the same team of experienced clinicians using a non-traumatic lumbar puncture procedure. Trained clinical psychiatrists performed clinical assessments, Glucose levels in CSF and serum from healthy subjects and schizophrenic patients were measured immediately after collection using a NOVA BioProfile analyser (Nova Biomedical, Waltham, USA). CSF samples were divided into aliquots and stored at −80° C. None of the samples underwent more than 2 freeze-thaw cycle prior to acquisition of NMR spectra. All experiments were performed under blind and randomized conditions. CSF samples (150 μl) were made up to a final volume of 500 μl by the addition of D₂O in preparation for ¹H NMR analysis.

¹H NMR Spectroscopy of CSF Samples: Standard 1-D 600 MHz ¹H NMR spectra were acquired for all samples using the first increment of the NOESY pulse sequence to effect suppression of the water resonance and limit the effect of B₀ and B₁ inhomogeneities in the spectra (pulse sequence: relaxation delay-90°-t₁-90°-tₘ-90°-acquire FID; Bruker Analytische GmbH, Rheinstetten, Germany). In this pulse sequence, a secondary radio frequency irradiation is applied at the water resonance frequency during the relaxation delay of 2 s and the mixing period (tₘ=100 ms), with t₁ fixed at 3 μs. Typically 256 transients were acquired at 300K into 32K data points, with a spectral width of 6000 Hz and an acquisition time of 1.36 s per scan. Prior to Fourier transformation, the free induction decays (FID's) were multiplied by an exponential weight function corresponding to the line-broadening of 0.3 Hz.

Data Reduction and Pattern Recognition Procedures: To efficiently evaluate the metabolic variability within and between biofluids derived from patients and controls, spectra were data reduced using the software program AMIX (Analysis of MIXtures version 2.5, Bruker Rheinstetten, Germany) and exported into SIMCA P (version 10.5, Umetrics AB, Umea, Sweden) where a range of multivariate statistical analyses were conducted. Initially principal components analysis (PCA) was applied to the data in order to discern the presence of inherent similarities in spectral profiles. Only one spectrum was excluded from the analysis on the basis of the Hotellings t-test which provided a 95% confidence value for a model based on the sample composition. Poor water suppression and high citrate composition were the main cause of sample exclusion. Where the classification of ¹H NMR spectra was influenced by exogenous contaminants, the spectral regions containing those signals were removed from statistical analysis. In order to confirm the biomarkers differentiating between the schizophrenia patients and matched controls, projection to latent structure discriminant analysis (PLS-DA) was employed. Orthogonal signal correction (OSC) of NMR data: The OSC method was used to remove variation in the data matrix between samples that is not correlated with the Y-vector [16]. The resulting data set was filtered to allow pattern recognition focused on the variation correlated to features of interest within the sample population, this improves the predictivity and separation power of pattern recognition methods.

Where appropriate, data were subjected to one-way analysis of variance (ANOVA) using the Statistical Package for Social Scientists (SPSS/PC+; SPSS, Chicago). Where the F ratio gave P<0.05, comparisons between individual group means were made by Tukey's test for post-hoc comparisons when the variance was equal between groups. Dunnett's T3 test was used for post-hoc comparisons if variances were not equal. The significance levels was set at p=0.05.

Plots of PLS-DA scores based on ¹H NMR spectra of CSF samples showed a clear differentiation between healthy volunteers and drug-naïve patients with first onset, paranoid schizophrenia (FIG. 1). The loading coefficients indicated that glucose, acetate, alanine and glutamine resonances were predominantly responsible for the separation between classes. Results from ¹H NMR spectroscopy showed significantly elevated glucose concentrations in CSF samples from first-onset, drug-naïve, paranoid schizophrenia patients as compared to the demographically matched control group, with a relative increase in concentration of 6.5%±0.94% (p=0.04, One-way ANOVA). Direct measurements of CSF glucose levels (performed immediately after sample collection) confirmed that glucose levels in drug-naïve schizophrenia patients in the first cohort were significantly higher than in healthy volunteers (6.5% increase, p=0.005; Table 1).

TABLE 1
Demographic details, CSF and serum glucose levels of subjects

| | | Drug Naive | Drug Naive | Schizophrenia | Schizophrenia |
|---|---|---|---|---|---|
| | | Paranoid | Paranoid | treated | treated with |
| | Healthy | Schizophrenia | Schizophrenia | with “typical” | “atypical” |
| | Volunteer | (PS, | (PS 2^{nd} | antipsychotic | antipyschotic |
| | (HV) | 1^{st} cohort) | cohort) | (ST) | (SAT) |
| | (n = 70) | (n = 37) | (n = 17) | (n = 6) | (n = 22) |
| Age (yrs)^{#} | 27.4 ± 5.9 | 28.1 ± 9.4 | 25.0 ± 5.6 | 31.5 ± 5.5 | 29.2 ± 10.1 |
| Sex^{&} | | | | | |
| male | 39 | 27 | 12 | 5 | 17 |
| female | 31 | 10 | 5 | 1 | 5 |
| [Glucose](mg/dl) | | | | | |
| CSF | 58.5 ± 4.6* | 62.3 ± 5.5 | 65.3 ± 6.4 | 65.0 ± 5.9 | 64.9 ± 6.4 |
| Serum | 87.2 ± 15.0** | 93.1 ± 14.4 | 91.5 ± 9.9 | 87.3 ± 19.2 | 103.5 ± 24.7 |
| Duration of | N/A | N/A | N/A | 9.6 ± 8.3 | 9.2 ± 6.2 |
| treatment (days) | | | | | |

^{#}There is no significant difference in age between the control and disease groups (Oneway-ANOVA).
^{&}Female gender is over-represented in the HV group, but sex appears to have no effect on CSF metabolite profiles (see FIG. 5).
*Glucose levels in CSF from healthy volunteers (HV) are lower than the glucose levels in CSF from drug-naive paranoid schizophrenia patients (PS), paranoid schizophrenia patients treated with typical (ST) and atypical (SAT) anti-psychotic medication (HV vs. PS (two cohorts included), p < 0.001; HV vs. SAT, p < 0.001; HV vs. ST, p = 0.02, One-way ANOVA with Tukey's test).
**Serum glucose levels are significantly increased only in schizophrenia patients treated with atypical anti-psychotics (HV vs. SAT, p = 0.05, One-way ANOVA with Dunnett's T3 test). There is no significant difference in serum glucose level between other groups.
All data are shown in mean ± s.d.

Interestingly, serum glucose levels obtained from the same schizophrenia and healthy subjects showed no difference (p=0.24), suggesting a brain/CSF-specific elevation in glucose levels. In contrast, acetate and lactate concentrations were reduced (11.5%, p=0.006; and 17.3%, p=0.05 (t test), respectively) in drug-naïve schizophrenia patients (the first cohort) compared to matched controls. Spectral changes corresponding to glutamine and alanine resulted from a pH dependent change in the chemical shift of these resonances. The pH of CSF samples from untreated schizophrenia patients was found to be on average 0.1 pH units lower than in the matched control samples (p<0.05, t test) which corresponded to a mean chemical shift change of 0.015 ppm for the β-CH₂ resonance of glutamine and 0.016 ppm shift change for the alanine CH₃ signal. Short term treatment for an average of nine days (see Table 1) with atypical anti-psychotic medication resulted in a normalisation of the CSF metabolite profile in approximately 50% of the schizophrenia patients (FIG. 2A), whereas treatment with typical anti-psychotic medication did not show such an effect (FIG. 2A), although as the number of patients treated with typical anti-psychotics is low (n=6), no clear conclusions can be drawn from this observation. Interestingly, it was observed that patients who suffered several psychotic episodes before drug treatment was initiated (either with typical or atypical anti-psychotics) did not show a normalisation of their CSF disease profile over the duration of the study. Six out of a total of seven patients with more than one episode before drug treatment clustered closely with the drug-naïve schizophrenia group and, indeed, none of them clustered with the healthy control group (FIG. 2B). Moreover, all schizophrenia patients who exhibited a normalisation of the CSF metabolite profile (either with typical or atypical anti-psychotics) had commenced medication during their first psychotic presentation. In statistical terms (recognising that numbers are small), this study implies that if treatment is initiated during a first episode, 57% of patients recover (assessed in terms of normalisation of CSF metabolite profiles), whilst if medication was given after a second psychotic episode, no normalisation (0/7) was observed within the time frame of this study.

Due to the prevalent cannabis use amongst schizophrenia patients and the known influence of cannabis on glucoregulation, the influence of this potential confounding factor was examined in the disease and control groups. None of the control patients had tested positive on urine drug screen and no change in CSF metabolites was observed between healthy volunteers who reported moderate (>5 times/lifetime) or low/no (<2 times/lifetime) cannabis use (data not shown). In the drug naïve, paranoid schizophrenia group, 7 patients (out of a total of 37) tested positive for cannabis on urine drug screen. Cannabis positive patients had significantly lower serum glucose levels (9% decrease; p=0.05, t test), but no effect on CSF glucose levels was observed (p=0.20, t test; see FIG. 6 and Table 2). Three patients who tested positive for cannabis were found to have highly altered CSF metabolite profiles and formed a separate cluster in the PLS-DA plot (away from both healthy controls and schizophrenia patients) whilst the remaining four cannabis positive patients clustered with the drug negative group (see FIG. 6).

TABLE 2
Effect of cannabis use on serum and CSF glucose
levels in paranoid schizophrenia patients.

| | Paranoid schizophrenia | Paranoid schizophrenia |
|---|---|---|
| | patients with cannabis | patients with cannabis |
| | “positive” in urine | “negative” in urine |
| | (n = 7) | (n = 30) |
| CSF glucose | 60.3 ± 4.3 | 62.9 ± 5.7 |
| concentration | | |
| Serum glucose | 86.3 ± 9.0 | 95.1 ± 15.3* |
| concentration | | |

Data are shown as mean ± S.D.
Data are shown as mean * p = 0.05, t test.

Validation of key metabolic alterations in an independent test sample set. To validate the findings, samples from the first cohort (70 control and 37 first onset, drug naïve schizophrenia CSF samples), were re-analyzed alongside a second cohort of 17 additional first onset, drug naïve schizophrenia patients. A model was built based on a training set of 50 randomly selected control samples and 37 first onset, drug naïve schizophrenia samples from the first cohort. Both PCA and PLS-DA showed similar results as shown in FIG. 1 (FIG. 4). This model was then used to predict class membership in a test set comprising of 20 control CSF samples (from the first cohort) and 17 first onset, drug naïve schizophrenia patients (from the 2nd cohort, Table 2). Orthogonal signal correction (OSC) was applied to enhance the metabolic differentiation between classes within the model [4]. After OSC, the separation of control and first onset, drug naïve schizophrenia groups in the PLS scores plots (FIG. 3A) was characterized by similar spectral regions to those previously identified as contributing to the separation of the classes, i.e. glucose, lactate, shifts in glutamine resonances and citrate (FIG. 3B). The PLS model calculated from OSC-filtered NMR data was then used to predict class membership in the test sample set. The Y-predicted scatter plot assigned samples to either to the control or schizophrenia group using an a priori cut-off of 0.5, and showed the ability of ¹H-NMR metabonomics analysis to predict class membership of unknown samples with a sensitivity of 82% and a specificity of 85% (FIG. 3C).

Analysis of the ¹H NMR spectra of CSF samples showed a differential distribution of samples from healthy volunteers away from drug-naïve patients with first onset schizophrenia (FIGS. 1B and 1C). The metabolic profile of CSF was found to be characteristically altered in schizophrenia patients and the majority of key metabolites contributing to the separation were replicated in an independent test set (FIG. 3). There was some overlap of the two sample classes in the PLS-DA scores plot derived from the NMR spectra (FIGS. 1B and 1C). Whilst the drug naïve, paranoid schizophrenia group clustered very tightly together, a small number of samples did not show a clear separation in the PLS-DA analysis. This may indicate the existence of schizophrenia sub-groups; also clinical parameters, such as disease progression, severity and/or drug-response may relate to distinct metabolic signatures. Although the sample size of this study was too small to enable strong conclusions about patient subgroups to be drawn, it was of interest that all 4 patients who were found to cluster with the control group (FIG. 1B), had an exceptionally good outcome or recovered fully from a first episode of psychosis.

Abnormal glucose levels in serum have been linked with anti-psychotic drug treatment [17,18], yet the observations made in this study of an elevation of CSF glucose concentrations in schizophrenia patients imply that glucoregulatory alterations are intrinsic to the schizophrenia syndrome and are brain-specific, because samples collected from drug-naïve, first onset patients showed significantly increased CSF glucose levels and glucose elevation was not observed in sera from the same schizophrenia subjects. Elevated CSF glucose has not previously been reported for schizophrenia, however abnormal fasting glucose tolerance has been observed in serum from first-onset patients [19]. The prevalence of diabetes type II is substantially increased in schizophrenia patients (15.8% as compared to 2-3% in the general population) [20]. Studies have also found increased plasma levels of glucose and norepinephrine in schizophrenia patients [21-23] although increased serum glucose and the high prevalence of type II diabetes in schizophrenic patients have mainly been attributed to anti-psychotic drug treatment [17,23]. Indeed, in this study, serum glucose levels were found to be increased in patients treated with atypical anti-psychotic medication (Table 1). It is possible that drug treatment precipitates the onset of diabetes in schizophrenia patients in the context of a co-predisposition and that both schizophrenia and diabetes type II share common disease mechanisms. The significantly lower CSF pH observed aligns with observations in post-mortem brain and may be attributed to alterations in energy metabolism at large [24]. Numerous other studies on post-mortem brain have also found mitochondrial changes in schizophrenia (e.g. [25,26]). The lowered pH observed in CSF in this study may thus be due to alterations in cellular respiration. Surprisingly, however, whilst an increase in lactate in postmortem brain tissue has been found, in this study a significant decrease in CSF lactate levels was detected in first onset schizophrenia patients. At this stage it is not possible to determine which metabolite alterations are contributing to the lowered pH in CSF. A possible explanation could be that the “schizophrenia brain” preferentially utilizes lactate over glucose as energy substrate. Brain lactate is believed to be predominantly produced by astrocytes [27] and is used as energy substrate in brain, in particular by neurons under certain conditions [27]. In fact, significant monocarboxylate utilization by the brain was also reported in different pathological states such as diabetes and prolonged starvation [28,29].

Acetate was also found to be significantly reduced in the CSF of first-onset, drug naïve schizophrenia patients. The majority of acetate in the brain is utilised in fatty acid and lipid synthesis [30], thus the decreased acetate concentration may suggest a compromised synthesis of myelin-related fatty acids and lipids in the schizophrenia brain. Acetate in the brain is primarily derived from N-acetylaspartate (NAA), which is hydrolyzed into L-aspartate and acetate by the enzyme aspartoacylase (ASPA) [31]. NAA is synthesized in neuronal mitochondria and transferred to oligodendrocytes, where ASPA liberates the acetate moiety to be used for myelin lipid synthesis [32]. An in vivo reduction in NAA levels in schizophrenia is a well-established observation [33]. More interestingly, we found ASPA transcripts down-regulated in post-mortem brain using microarray and quantitative PCR (Q-PCR) analysis in schizophrenia post-mortem brain (−1.78; p=0.09 by microarray; −1.61; p=0.04 by Q-PCR; n=15 schizophrenia prefrontal cortex and matched controls; unpublished). Together with our findings of a significant decrease of acetate in CSF, this lends further support not only for altered NAA metabolism, but also for oligodendrocyte dysfunction, which we and others previously reported [34,35].

Perturbations in CSF acetate concentrations have also been observed in patients with CJD, although in contrast to the current study, CJD was associated with an increase in acetate concentrations [36]. Disturbed glucose metabolism has also been associated with mood and psychotic disorders [37], although to our knowledge none of these studies measured CSF glucose levels. However, the increased concentrations of glucose together with other metabolic perturbations, such as lower levels of acetate and lactate, and a pH-dependent shift in glutamine resonances, may represent a more specific disease diagnostic for schizophrenia.

The effects of two drug treatment regimen, the use of typical and atypical anti-psychotic medication, were evaluated using the same analytical methods.

Normalization of the metabolite profiles was observed in patients (n=28) who had been treated with atypical anti-psychotic medication for an average of 9 days. FIG. 2 illustrates a shift of approximately 50% of patients on atypical anti-psychotics towards the cluster of healthy controls within the PLS-DA plot. These results are indicative that atypical medication results in a normalization of the metabonomic disease signature. It is a well-established fact that only between 50-70% (according to different sources) of schizophrenia patients respond to anti-psychotic intervention. However, clinical response is generally only observed after weeks or months of treatment. It is believed that normalization of the metabonomic signature detected in this study is liable to be predictive of clinical drug response.

One of the most striking findings of this study is the effect of number of psychotic episodes prior to commencing anti-psychotic treatment on CSF metabolite profile in paranoid schizophrenia patients. 57% of patients who were commenced on anti-psychotic medication during their first psychotic episode were found to cluster with the healthy control cluster whereas six out of the seven patients who had several psychotic episodes prior to treatment clustered with the drug-naïve, paranoid schizophrenia group (FIG. 2B). These results suggest that the initiation of anti-psychotic treatment during a first psychotic episode may influence treatment response or indeed outcome. This view is in agreement with The Personal Assessment and Crisis Evaluation (PACE) clinic study [38], the Prevention through Risk Identification, Management and Education (PRIME) study [39] and other ongoing studies that purport that early identification of patients at risk of developing schizophrenia with subsequent intervention may reduce morbidity and adverse outcome. Metabonomic approaches to profiling CSF employed in this study provide a new approach to achieving both early diagnosis and monitoring therapeutic intervention for schizophrenia.

As many schizophrenia patients are recreational cannabis users and as cannabis has a known effect on glucoregulation, this potential confounding factor was examined. Recent cannabis use was associated with a significant reduction in serum glucose, but no influence on the CSF metabolite profile was observed.

The application of metabolite profiling tools as described herein provides an efficient means for early diagnosis of psychotic disorders such as paranoid schizophrenia and provides a practical method for monitoring therapeutic intervention by providing metrics for the normalization of biofluid spectra by multivariate comparison with the relevant control profiles.

### REFERENCES

1. Nicholson J K, Lindon J C, Holmes E (1999) ‘Metabonomics’: understanding the metabolic responses of living systems to pathophysiological stimuli via multivariate statistical analysis of biological NMR spectroscopic data. Xenobiotica 29: 1181-1189.
2. Tsang T M, Griffin J L, Haselden J, Fish C T Holmes E (2005) Metabolic characterization of distinct neuroanatomical regions in rats by magic angle spinning (1)H nuclear magnetic resonance spectroscopy. Magn Reson Med 53: 1018-1024.
3. Nicholson J K, Connelly J, Lindon J C, Holmes E (2002) Metabonomics: a platform for studying drug toxicity and gene function. Nat Rev Drug Discov 1: 153-161.
4. Brindle J T, Antti H, Holmes E, Tranter G, Nicholson J K, et al. (2002) Rapid and noninvasive diagnosis of the presence and severity of coronary heart disease using 1H-NMR-based metabonomics. Nat Med 8: 1439-1444.
5. Nicholson J K, Holmes E, Lindon J C, Wilson I D (2004) The challenges of modeling mammalian biocomplexity. Nat Biotechnol 22: 1268-1274.
6. Cheng L L, Newell K, Mallory A E, Hyman B T, Gonzalez R G (2002) Quantification of neurons in Alzheimer and control brains with ex vivo high resolution magic angle spinning proton magnetic resonance spectroscopy and stereology. Magn Reson Imaging 20: 527-533.
7. Cheng L L, Ma M J, Becerra L, Ptak T, Tracey I, et al. (1997) Quantitative neuropathology by high resolution magic angle spinning proton magnetic resonance spectroscopy. Proc Natl Acad Sci USA 94: 6408-6413.
8. Beckwith-Hall B M, Nicholson J K, Nicholls A W, Foxall P J, Lindon J C, et al. (1998) Nuclear magnetic resonance spectroscopic and principal components analysis investigations into biochemical effects of three model hepatotoxins. Chem Res Toxicol 11: 260-272.
9. Holmes E, Foxall P J, Spraul M, Farrant R D, Nicholson J K, et al. (1997) 750 MHz 1H NMR spectroscopy characterisation of the complex metabolic pattern of urine from patients with inborn errors of metabolism: 2-hydroxyglutaric aciduria and maple syrup urine disease. J Pharm Biomed Anal 15: 1647-1659.
10. Garseth M, Sonnewald U, White L R, Rod M, Nygaard O, et al. (2002) Metabolic changes in the cerebrospinal fluid of patients with lumbar disc herniation or spinal stenosis. J Neurosci Res 69: 692-695.
11. Braun K P, Gooskens R H, Vandertop W P, Tulleken C A, van der Grond J (2003) 1H magnetic resonance spectroscopy in human hydrocephalus. J Magn Reson Imaging 17: 291-299.
12. Koschorek F, Offermann W, Stelten J, Braunsdorf W E, Steller U, et al. (1993) High-resolution 1H NMR spectroscopy of cerebrospinal fluid in spinal diseases. Neurosurg Rev 16: 307-315.
13. Hashimoto K, Engberg G, Shimizu E, Nordin C, Lindstrom L, et al. (2005) Elevated glutamine/glutamate ratio in cerebrospinal fluid of first episode and drug naïve schizophrenic patients. BMC Psychiatry 5: 1-6.
14. White L R, Garseth M, Aasly J, Sonnewald U (2004) Cerebrospinal fluid from patients with dementia contains increased amounts of an unknown factor. J Neurosci Res 78: 297-301.
15. Do K Q, Trabesinger A H, Kirsten-Kruger M, Lauer C J, Dydak U, et al. (2000) Schizophrenia: glutathione deficit in cerebrospinal fluid and prefrontal cortex in vivo. Eur J Neurosci 12: 3721-3728.
16. Wold S, Antti H, Lindgren F, Ohman J (1998) Orthogonal signal correction of near-infrared spectra. Chemometrics Intelligent Lab Systems 44: 175-185.
17. Henderson D C, Cagliero E, Copeland P M, Borba C P, Evins E, et al. (2005) Glucose metabolism in patients with schizophrenia treated with atypical anti-psychotic agents: a frequently sampled intravenous glucose tolerance test and minimal model analysis. Arch Gen Psychiatry 62: 19-28.
18. Newcomer J W (2004) Abnormalities of glucose metabolism associated with atypical anti-psychotic drugs. J Clin Psychiatry 65 Suppl 18: 36-46.
19. Ryan M C, Collins P, Thakore J H (2003) Impaired fasting glucose tolerance in first-episode, drug-naïve patients with schizophrenia. Am J Psychiatry 160: 284-289.
20. Henderson D C, Ettinger E R (2002) Schizophrenia and diabetes. Int Rev Neurobiol 51: 481-501.
21. Arranz B, Rosel P, Ramirez N, Duenas R, Fernandez P, et al. (2004) Insulin resistance and increased leptin concentrations in noncompliant schizophrenia patients but not in anti-psychotic-naïve first-episode schizophrenia patients. J Clin Psychiatry 65: 1335-1342.
22. Dinan T, Peveler R, Holt R (2004) Understanding schizophrenia and diabetes. Hosp Med 65: 485-488.
23. Elman I, Rott D, Green A I, Langleben D D, Lukas S E, et al. (2004) Effects of pharmacological doses of 2-deoxyglucose on plasma catecholamines and glucose levels in patients with schizophrenia. Psychopharmacology (Berl) 176: 369-375.
24. Prabakaran S, Swatton J, Ryan M, Huffaker H, Huang T J, et al. (2004) An integrative functional genomics approach reveals impaired brain energy metabolism in Schizophrenia. Mol Psychiatry: (in press).
25. Iwamoto K, Bundo M, Kato T (2005) Altered expression of mitochondria-related genes in postmortem brains of patients with bipolar disorder or schizophrenia, as revealed by large-scale DNA microarray analysis. Hum Mol Genet 14: 241-253.
26. Karry R, Klein E, Ben Shachar D (2004) Mitochondrial complex I subunits expression is altered in schizophrenia: a postmortem study. Biol Psychiatry 55: 676-684.
27. Pierre K, Pellerin L (2005) Monocarboxylate transporters in the central nervous system: distribution, regulation and function. J Neurochem 94: 1-14.
28. Hawkins R A, Mans A M, Davis D W (1986) Regional ketone body utilization by rat brain in starvation and diabetes. Am J Physiol 250: E169-178.
29. Fernandes J, Berger R, Smit GP (1982) Lactate as energy source for brain in glucose-6-phosphatase deficient child. Lancet 1: 113,
30. Kammula R G, Fong BC (1973) Metabolism of glucose and acetate by the ovine brain in vivo. Am J Physiol 225: 110-113.
31. Madhavarao C N, Arun P, Moffett J R, Szucs S, Surendran S, et al. (2005) Defective N-acetylaspartate catabolism reduces brain acetate levels and myelin lipid synthesis in Canavan's disease. Proc Natl Acad Sci USA 102: 5221-5226.
32. Chakraborty G, Mekala P, Yahya D, Wu G, Ledeen R W (2001) Intraneuronal N-acetylaspartate supplies acetyl groups for myelin lipid synthesis: evidence for myelin-associated aspartoacylase. J Neurochem 78: 736-745.
33. Steen R G, Hamer R M, Lieberman J A (2005) Measurement of brain metabolites by 1H magnetic resonance spectroscopy in patients with schizophrenia: a systematic review and meta-analysis. Neuropsychopharmacology 30: 1949-1962.
34. Prabakaran S, Swatton J E, Ryan M M, Huffaker S J, Huang J T, et al. (2004) Mitochondrial dysfunction in schizophrenia: evidence for compromised brain metabolism and oxidative stress. Mol Psychiatry 9: 684-697, 643.
35. Hakak Y, Walker J R, Li C, Wong W H, Davis K L, et al. (2001) Genome-wide expression analysis reveals dysregulation of myelination-related genes in chronic schizophrenia. Proc Natl Acad Sci USA 98: 4746-4751.
36. Maillet S, Vion-Dury J, Confort-Gouny S, Nicoli F, Lutz NW, et al. (1998) Experimental protocol for clinical analysis of cerebrospinal fluid by high resolution proton magnetic resonance spectroscopy. Brain Res Brain Res Protoc 3: 123-134.
37. Regenold W T, Phatak P, Kling M A, Hauser P (2004) Post-mortem evidence from human brain tissue of disturbed glucose metabolism in mood and psychotic disorders. Mol Psychiatry 9: 731-733.
38. McGorry P D, Yung A R, Phillips L J, Yuen H P, Francey S, et al. (2002) Randomized controlled trial of interventions designed to reduce the risk of progression to first-episode psychosis in a clinical sample with sub threshold symptoms. Arch Gen Psychiatry 59: 921-928.
39. McGlashan T H. Abstract presented at the Twelfth Biennial Winter Workshop on Schizophrenia. In: Davos, editor; 2004. Switzerland.
40. Geladi, P., and B. R. Kowalski (1986), “Partial Least Squares Regression: A Tutorial,” Analytica Chimica Acta, 185, 1-17.

1-53. (canceled)

54. A method of confirming or monitoring a psychotic disorder in a subject, comprising measuring the level of one or more biomarkers selected from glucose, lactate, acetate species and pH, in a sample of cerebrospinal fluid (CSF) taken from the subject.

55. The method of claim 54 which is used to monitor the efficacy of a therapy in a subject having a psychotic disorder.

56. The method according to claim 55, comprising comparing the level of one or more biomarkers with the level of the one or more biomarkers in one or more samples taken from the subject prior to commencement of the therapy.

57. The method according to claim 55, wherein the therapy is an anti-psychotic disorder therapy.

58. The method according to claim 54, comprising measuring the levels of the one or more biomarkers in CSF samples taken on two or more occasions.

59. The method according to claim 58, comprising comparing the levels of the one or more biomarkers.

60. The method according to claim 54, comprising comparing the level of the one or more biomarkers in a CSF sample with the level of the one or more biomarkers in one or more controls.

61. The method according to claim 60, wherein the one or more controls are a normal control and/or a psychotic disorder control.

62. The method according to claim 54, comprising quantifying the one or more biomarkers in a further biological sample taken from the test subject.

63. The method according to claim 62, wherein the further biological sample is selected from whole blood, blood serum, urine, saliva, or other body fluid, or breath, condensed breath, or an extract or purification therefrom, or dilution thereof.

64. The method according to claim 54, wherein the or each level is detected by analysis of NMR spectra.

65. The method according to claim 54, wherein the or each level is detected by a method selected from NMR, SELDI (−TOF) and/or MALDI (−TOF), 1-D gel-based analysis, 2-D gel-based analysis, mass spectrometry (MS) and LC-MS-based technique.

66. The method according to claim 54, wherein the or each level is detected by a method selected from direct or indirect, coupled or uncoupled enzymatic methods; and electrochemical; spectrophotometric; fluorimetric; luminometric; spectrometric; polarimetric; and chromatographic techniques.

67. The method according to claim 54, wherein the or each level is detected using a sensor or biosensor comprising one or more enzymes; binding, receptor, or transporter proteins; synthetic receptors; or other selective binding molecules, for direct or indirect detection of the one or more biomarkers, said detection being coupled to an electrical, optical, acoustic, magnetic or thermal transducer.

68. The method according to claim 54, wherein the psychotic disorder is a schizophrenic disorder.

69. The method according to claim 68, wherein the schizophrenic disorder is selected from paranoid, catatonic, disorganised, undifferentiated and residual schizophrenia.

70. The method according to claim 54, wherein the psychotic disorder is a bipolar disorder.

71. A method of identifying a substance capable of modulating a psychotic disorder in a subject, comprising administering a test substance to a test subject, and detecting the level of one or more biomarkers selected from glucose, lactate, acetate species and pH, in a CSF sample taken from the subject.
